(19) United States
(12) Patent Application Publication
(10) Pub. No.: US 2014/0134572 A1
(43) Pub. Date: May 15, 2014

(54) Polymeric Material for Taking a Dental Impression and Method Thereof

(71) Applicant: COLTENE AG, Altstatten (CH)

(72) Inventors: Stephan Lampl, Luchingen (CH); Dierk Lubbers, Eichberg (CH); Ralf Kollefrath, Ruethi (CH); Manfred Kalt, Altstatten (CH)

(73) Assignee: COLTENE AG, Altstatten (CH)

(21) Appl. No.: 14/157,787

(22) Filed: Jan. 17, 2014

Related U.S. Application Data
(63) Continuation-in-part of application No. 11/637,204, filed on Dec. 11, 2006, now abandoned.
(60) Provisional application No. 60/750,624, filed on Dec. 15, 2005.

(51) Int. Cl. A61K 6/10; A61C 9/00

(52) U.S. Cl. CPC A61K 6/10; A61C 9/0006
USPC 433/214; 433/215

(57) ABSTRACT

A polymeric material is curable from an uncured state to a cured state and includes at least one finely dispersed filler. The material has in the cured state, a translucency in the range of about 30% to about 100%, and shore A hardness according to DIN 53505:1984 in the range of about 20 to about 70 and a tensile strength according to DIN 53504:1994 in the range of about 1.5 MPa to about 4.5 MPa. The material has in the uncured state, a consistency according to DIN ISO 4823:2000 of type 0 to 3.

### CROSS-REFERENCE TO RELATED APPLICATIONS

This application is a continuation-in-part of and claims priority to U.S. Ser. No. 11/637,204, which was filed Dec. 11, 2006 and which is hereby incorporated by reference in its entirety for all purposes.

U.S. Ser. No. 11/637,204 is a non-provisional counterpart and claims priority to U.S. Ser. No. 60/750,624, which was filed Dec. 15, 2005 and which is hereby incorporated by reference in its entirety for all purposes.

### FIELD OF THE INVENTION

The present invention to materials used in dentistry, and specifically to a polymeric material suitable for taking an dental impression, kits including the polymeric material, dental impressions including the polymeric material, and method of use of the polymeric material.

### BACKGROUND OF THE INVENTION

In the course of various dental applications, e.g. in dental restoration, it is necessary to take an impression of the patient's dental situation, in order to provide the dentist with a 3D model. Such a 3D model needs to resemble the dental situation as exactly as possible in order to provide the dentist with a suitable basis e.g. for a satisfying restoration being prepared, such as a crown, a bridge, or the like.

A common problem in dental impression taking is a loss in precision of the mold due to irregularities, e.g. inclusion of air bubbles within or under the impression material, resulting in non-molded areas, which are not reproduced. Such non-molded areas are afterwards either extrapolated by the dentist, if possible (often resulting in non-satisfactory results) or the impression taking must be performed once again in the worst case, causing inconvenience to both the patient and the dentist. In the state of the art, inclusion of air bubbles within or under the impression material was tried to be eliminated by various approaches, e.g. by modifying the flow-characteristics of the impression material, or by using special impression trays, which aims to eliminate such air inclusions by applying reduced pressure to the impression region. However, all these approaches do not allow for a reliable elimination of said air inclusions under all circumstances, and/or afford complicated impression tray devices.

### BRIEF SUMMARY OF THE INVENTION

It is an object of the present invention to overcome the drawbacks of the prior art, especially to more reliably allow for the elimination of irregularities, e.g. the inclusion of air bubbles within and/or under an impression material, in order to more reliably allow for a highly detailed impression taking.

This object has been solved, in one aspect, by a method of dental impression taking and a polymeric material therefor, as outlined below.

According to one aspect of the invention, a method of dental impression taking comprises the step of applying a polymeric material at least partially to the region to be reproduced by the impression, wherein the polymeric material is translucent. Preferably, the translucency of the polymeric material is in the range from about 30% to about 100%, preferably ≧40%, and most preferably ≧50%.

The method of impression taking comprises the additional steps of:
identifying an irregularity, especially air inclusion(s) within and/or under said translucent polymeric material; and
eliminating said irregularities before hardening of said translucent polymeric material.

### DETAILED DESCRIPTION OF THE INVENTION

The use of a polymeric, translucent material in dental impression taking is not previously known. In contrast, commonly used impression materials such as silicone materials are opaque, mostly due to (especially relatively high contents of) filling materials. However, according to the invention, the above-mentioned drawbacks of the prior art have surprisingly been overcome by using a translucent impression material, because a sufficient translucency allows for easy identification of possible air inclusion(s). Such identified air inclusion(s) can then easily be eliminated before hardening of the applied impression material, e.g. by simply sticking and/or slightly turning the application tip of common dispensers of impression material at the air inclusion(s), whereupon the air inclusion(s) can be easily eliminated.

Preferably, the process of dental impression taking according to the invention is a process chosen from the group consisting of two-material-two-phase processes, two-material-one-phase processes, and one-material-one-phase processes. These techniques are known by those skilled in the art. According to the two-material-two-phase process a crude impression is taken with a kneadable or heavy body impression material, which is subsequently additionally manipulated, e.g. with cutting instruments outside the patient's mouth, and finally a correction impression material is applied onto said manipulated crude impression again into the patient's mouth to a final impression mold. According to the invention, a translucent impression material is used at least as the correction impression material; if wanted and found appropriate in a special case, both impression materials may be translucent.

According to the two-material-one-phase process, two impression materials are applied at the same time, the one afar from the tooth and the other adjacent to the tooth. Further according to the invention, a translucent impression material is used adjacent to the tooth; if wanted and found appropriate in a special case, both impression materials may be translucent.

According to the one-material-one-phase process, one and the same material is applied in an impression tray and additionally e.g. with a syringe. According to the invention, a translucent impression material is used.

According to the invention, there is provided a polymeric material for dental applications, especially for impression taking, characterized in that it exhibits the following features in its cured state:
a translucency in the range of about 30% to about 100%, preferably ≧40%, and most preferably ≧50%;
a shore A hardness according to DIN 53505:1987 in the range of about 20 to about 70, preferably from about 30 to about 60, and most preferably from about 45 to 55;
a tensile strength according to DIN 53504:1994 in the range of about 0.2 MPa to about 7 MPa, preferably from about 1 MPa to about 6 MPa, and most preferably from about 1.5 MPa to about 5.5 MPa.

Translucency in the above-mentioned ranges has been proven sufficient, the high translucencies being ideal for visual control of the applied material for the presence of air bubbles. The sample is prepared by filling the uncured material in a stainless steel form of 25 mm*20 mm*1 mm, and pressing off excessive material with a glass plate. After curing at 23° C., the sample is taken out.

Translucency of the polymeric material is determined through the 1 mm dimension of the sample with a BaSO₄ white background in a US/VIS spectrophotometer (LAMBDA 16, Perkin Elmer) with “ULBRICHTscher Kugel”. The background correction is measured against a BaSO₄ white standard.

The above-mentioned ranges of shore A hardness according to DIN 53505:1987 and tensile strength according to DIN 53504:1994 have proven excellent for putting into practice the materials according to all embodiments of the present invention. A person skilled in the art can easily choose and adapt the shore A hardness and the tensile strength in the above-mentioned ranges, by routine laboratory techniques, e.g. incorporation of suitable additives which do not hamper the above-mentioned translucency. If, for example, fillers are needed to be incorporated, they must be accordingly chosen (i.e., suitably finely dispersed and/or having a suitable refractive index) and in such an amount that the translucency requirement is fulfilled.

In order to avoid any doubt, a material comprising polymers and fillers, in particular inorganic fillers, is also regarded as a “polymeric material” within the context of the present application, as long as the weight content of polymers in the material is at least 10% by weight.

The material exhibits, in its uncured state, a consistency according to DIN ISO 4823:2000 of type 0 to type 3, preferably of type 2 or type 3.

Currently, hydrophilic impression materials are preferred in the art. Accordingly, the material according to the invention preferably exhibits a wetting angle of contact of less than about 50° after 2 minutes. However, the invention is not limited to hydrophilic materials, i.e. angles of contact of more than about 50° after 2 minutes, especially of more than about 90° after 2 minutes may also be appropriate for some applications.

The angle of contact is determined as follows: a polymeric sample is prepared in a brass frame of 65 mm*25 mm*3 mm size and cured therein for about 10 minutes. Five minutes after detaching the sample from the frame, a droplet of deionized water is dropped onto the sample surface, and the angle of contact is determined with a drop shape analysis system DSA 10 of KRUSS GmbH, Hamburg, Germany. If so desired, the person of routine skill in the art will easily achieve and/or fine tune a suitable angle of contact e.g. by incorporating surfactants of common practice in the art, e.g. branched-nonylphenol ethoxylate (IGEPAL BC4), etc.

According to an especially preferred embodiment of the present invention, the polymeric material is a silicone-based material, preferably an addition-crosslinked silicone material. It is understood that condensation-crosslinked silicone materials are also appropriate.

As used here and henceforth, the term “addition-crosslinked” or “addition-crosslinkable” means that the polymer comprises at least one functional group which may react with a crosslinking agent via an addition reaction. A typical example is that the polymer comprises at least one vinyl group, preferably two vinyl groups, which may undergo an electrophilic addition reaction with an appropriate crosslinking agent. Preferably, these vinyl groups are terminal.

As used here and henceforth, the term “condensation-crosslinked” or “condensation-crosslinkable” means that the polymer comprises at least one functional group which may react with a crosslinking agent via a condensation reaction. A typical example is that the polymer comprises at least one hydroxyl group, preferably two hydroxyl groups, which may undergo a condensation reaction with an appropriate crosslinking agent, for example, a crosslinking agent comprising alkoxy silicates.

More precisely, the polymer contained in the polymeric material is or comprises a polyorganosiloxane, comprising building blocks suitably chosen from (but not necessarily comprising all of them) [M] (R₃SiO_{1/2}), [D] (R₂SiO_{2/2}), [T] (RSiO_{3/2}), and Q (SiO_{4/2}). The polyorganosiloxane may be linear, branched, cyclic and/or preferably crosslinked. The polyorganosiloxane is preferably modified by hydrosilylation, i.e. the addition of silanes and/or (poly)(organo)siloxanes comprising Si—H bonds to unsaturated groups, e.g. the vinyl groups of the above-mentioned polyorganosiloxanes. A person skilled in the art will readily choose a suitable composition of a polyorganosiloxane in order to meet the above-defined functional requirements of the polymeric material.

Alternatively, the polymeric material may also be a polyether-based material. Especially preferred is an aziridine-crosslinked polyether material. It is evident to the one skilled in the art how to adjust the functional requirements as outlined above in the context of silicone-based material also for a polyether-based material.

Moreover, the molecular weight of the polymeric material is chosen such that the required shore A hardness and tensile strength are obtained. Optionally, additives such as rheology modifiers may be added for adjustment of the said parameters as it is known to those skilled in the art. Especially dyes or pigments (such as e.g. fluorescent pigments e.g. Lumilux Blau LZ (Omya AG), or glimmer pigments such as Timica Extra Bright 1500 (Mimox (LCW))) may be added in suitable amounts to the composition as long as the translucency and the other critical parameters as outlined above are not hampered.

Moreover, at least one filler, in particular at least one inorganic filler, is added to the composition which does not hamper the translucency and the other critical parameters as outlined above, e.g. at least one finely dispersed filler, preferably at least one nanofiller and/or at least one filler which inherently exhibits a suitable refractive index.

Within the present application, a nanofiller is understood as a filler containing particles and wherein at least 50% of the particles in the number size distribution have one or more external dimensions in the size range from 1 nm to 100 nm. This property can be determined, for example, with a “Zetasizer Nano ZS” analyzer, obtainable from Malvern Instruments Ltd., Enigma Business Park, Grovewood Road, Malvern, Worcestershire, UK. WR14 1XZ. Suitable nanofillers are, for example, available under the tradename Aerosil.

A refractive index of the filler is regarded as suitable when, at a temperature of 20° C. and/or at a wavelength of 589 nm, it lies in the range from 1.40 to 1.54, and/or when it differs from the refractive index of the remaining components of the polymeric material at a temperature of 20° C. and/or at a wavelength of 589 nm by at most 0.02. More preferably, the refractive index of the filler at a temperature of 20° C. and/or at a wavelength of 589 nm is lower than that of the remaining components of the polymeric material at a temperature of 20° C. and/or at a wavelength of 589 nm, wherein the difference lies in the range between 0.01 and 0.02. Due to the slight or completely absent difference in the refractive indices, visible light (at least at the given wavelength) is virtually not scattered and/or absorbed at all by the polymeric material, which provides the required translucency. The remaining components may comprise or may be, for example, a silicone-based polymer as described above.

In preferred embodiments, the filler is a metal fluoride, for example, an alkaline earth fluoride. The metal fluoride may be selected, for example, from the group consisting of MgF₂, LiF, CaF₂, BaF₂ or any combination thereof. Optionally, the filler may be encapsulated, for example by silanization. By such an encapsulation, the filler can be made inert.

Additionally or alternatively, at least one filler may be selected from the group consisting of glass, in particular borosilicate glass, quartz, silica gel, SiO₂ particles, in particular spherical SiO₂ particles, silicate, such as laminated alumosilicates (clays), ceramic or aluminum oxide, for example corundum, carbon nanotubes and nanofibers, ultradisperse diamonds (nanodiamonds), fullerenes, inorganic nanotubes, calcium carbonate, metallic nanoparticles, or any combinations thereof. For example, SiO₂ particles have a refractive index at room temperature of 1.46, glass one of 1.45 to 2.14, and borosilicate glass one of 1.50 to 1.55.

In order to provide for an appropriate difference between the filler and the remaining components of the polymeric composition, when the polymeric material comprises a silicone-based material, the refractive index of the silicone-based material may be adapted by an appropriate organic substitution. For example, the silicone-based material may be a methyl substituted and/or phenyl substituted and/or cyclohexyl-substituted silicone, as for example poly(dimethylsiloxane), polymethylphenylsiloxane or poly(dicyclohexyl)siloxane, or a combination thereof.

The refractive index of a silicone depends in particular on the organic substituents R¹, R² and R³ on the silicon atom and on the degree of branching of the silicone. Terminal groups of the silicone may be described as R¹R²R³SiO_{1/2}, linear groups as R¹R²SiO_{2/2} and branching groups as R¹R²SiO_{3/2}. R¹ and/or R² and/or R³ may be selected independently on each silicon atom. R¹, R² and R³ are in this case selected from a variety of organic substituents with different numbers of carbon atoms. The organic substituents may be at any desired ratio to one another in a silicone. As a rule, a substituent comprises 1 to 12, in particular 1 to 8, carbon atoms. R¹, R² and R³ are selected, for example, from methyl, ethyl, cyclohexyl or phenyl, in particular methyl and phenyl.

Organic substituents with many carbon atoms generally increase the refractive index, while smaller substituents result in a lower refractive index. A silicone rich in methyl groups may, for example, have a low refractive index, for example, of 1.40 to 1.44. A silicone which is, for example, rich in phenyl groups or cyclohexyl groups may, on the other hand, exhibit a higher refractive index.

Likewise, with other polymers than silicones, the refractive indices may be adjusted by selection of substituents and/or by hybrid materials, for example, silicone epoxy.

It is additionally possible to adjust the refractive index of by mixing different polymers. For example, the refractive index of a silicone material may be adjusted by mixing various silicones having different refractive indices. In this way, the polymer material may comprise or consist of a polymer mixture of silicones with different organic substituents. It is however also possible for a silicone copolymer to be produced from various monomers comprising different organic substituents, and thus for the refractive index of the polymer to be adapted accordingly. A mixture of various silicone copolymers with various refractive indices may also be used to adjust the refractive index of the polymer.

Many of the above-mentioned fillers have the further advantage that they increase the volumetric heat capacity of the polymeric material. This leads to an increased processing time in the mouth, which is a generally desired property in the context of taking dental impressions. As a general rule, the higher the density of a filler, the higher its volumetric heat capacity. For this reason, fillers having a density of at least 2.0 g/cm³ are generally preferred in the present invention.

The viscosity of the uncured material is suitably adjusted in the range of about 0.5 to about 500 Pa*s, preferably about to about 400 Pa*s, more preferably about 100 to about 300 Pa*s, as measured according to Brookfield. In any case, the rheology of the mixture to be applied is adjusted to allow for application by conventional dispensers, e.g. manually operated double chamber cartridges.

In yet another aspect of the present invention, there is provided a kit of parts, comprising a translucent polymeric material preferably and a further polymeric, preferably translucent material. Such a kit of parts can be manufactured and shipped as it is current state of the art with all one-phase and two-phase processes in dental impression taking as described above. According to the invention, however, at least one or both polymeric materials are translucent.

According to another aspect of the present invention, the material according to the invention is used for the preparation of a dental impression chosen from the group consisting of (i) a key for temporary or definitive composite crowns, telescope crowns or bridges; (ii) a key for composite facings or veneers; (iii) a positioning key for orthodontic brackets; (iv) a positioning key prior to insertion of a dental implant; (v) an implant template matrix; and (vi) a (pre)impression for build-up of anterior and posterior teeth in restorative dentistry.

All the above-mentioned applications may exploit the same inherent advantages of a translucent material: Firstly, irregularities, bubbles or the like can be easily identified and eliminated before hardening. Secondly, the translucency of the material also allows for light-hardening of a suitably chosen, light-curing further material subsequently filled into the impression (e.g. to prepare a final model) by simply irradiating through the translucent material.

The invention will now be explained in more detail by a detailed description of preferred embodiments. However, in no way is the invention limited to only these embodiments.

### EXAMPLES

### 1. Compositions

The following two-component polymeric silicone materials were prepared:

### Example 1

### “Light Body” (SiH/Vinyl: 1.94)

### Base Paste:

| | | |
|---|---|---|
| 70.00 g | Silopren Base Mixture P300 from GE Bayer | (0.05 mmol Vinyl/g) |
| 9.00 g | Silopren Crosslinker 4.3 from GE Bayer | (4.20 mmol SiH/g) |
| 2.00 g | Silopren Chain Extender TP 3359 from GE Bayer | (1.42 mmol SiH/g) |
| 9.00 g | Vinylsilicone VS 50 from Hanse Chemical, an alpha/omega- | (0.63 mmol Vinyl/g) |
| | Divinylpolydimethylsiloxan with 50 mPa*s | |
| 10.00 g | Vinylsilicone VS 10,000 from Hanse Chemical, an alpha/omega- | (0.05 mmol Vinyl/g) |
| | Divinylpolydimethylsiloxan with 10,000 mPa*s | |

### Catalyst Paste:

| | | |
|---|---|---|
| 70.00 g | Silopren Base Mixture P300 from GE Bayer | (0.05 mmol Vinyl/g) |
| 0.35 g | Catalyst preparation (90 weight % alpha/omega divinylpolydimethylsiloxan with | (0.53 mmol Vinyl/g) |
| | 1,000 mPa*s; 10 weight % catalyst complex Karstedt; corresponding to 4 weight % | |
| | pure Pt) | |
| 10.00 g | Vinylsilicone VS 50 from Hanse Chemical, an alpha/omega | (0.63 mmol Vinyl/g) |
| | Divinylpolydimethylsiloxan with 50 mPa*s | |
| 0.025 g | Inhibitor PTS-I 27 (DVTMDS) from Wacker Chemical | (10.75 mmol Vinyl/g) |
| 19.70 g | Vinylsilicone VS 10,000 from Hanse Chemical, an alpha/omega- | (0.05 mmol Vinyl/g) |
| | Divinylpolydimethylsiloxan with 10,000 mPa*s | |

### Example 2

### “Regular Body” (SiH/Vinyl: 1.87)

### Base Paste:

| | | |
|---|---|---|
| 40.00 g | Silopren Base Mixture P1,300 from GE Bayer | (0.06 mmol Vinyl/g) |
| 9.00 g | Silopren Crosslinker 4.3 from GE Bayer | (4.20 mmol SiH/g) |
| 2.00 g | Silopren Chain Extender TP 3359 from GE Bayer | (1.42 mmol SiH/g) |
| 9.00 g | Vinylsilicone VS 50 from Hanse Chemical, an alpha/omega- | (0.63 mmol Vinyl/g) |
| | Divinylpolydimethylsiloxan with 50 mPa*s | |
| 10.00 g | Vinylsilicone VS 10,000 from Hanse Chemical, an alpha/omega- | (0.05 mmol Vinyl/g) |
| | Divinylpolydimethylsiloxan with 10,000 mPa*s | |
| 30.00 g | Silopren Base Mixture P300 from GE Bayer | (0.05 mmol Vinyl/g) |

### Catalyst Paste:

| | | |
|---|---|---|
| 40.00 g | Silopren Base Mixture P1,300 from GE Bayer | (0.06 mmol Vinyl/g) |
| 0.30 g | Catalyst preparation (90 weight % alpha/omega divinylpolydimethylsiloxan | (0.53 mmol Vinyl/g) |
| | with 1,000 mPa*s; 10 weight % catalyst complex “Karstedt”; corresponding | |
| | to 4 weight % pure Pt) | |
| 10.00 g | Vinylsilicone VS 50 from Hanse Chemical, an alpha/omega- | (0.63 mmol Vinyl/g) |
| | Divinylpolydimethylsiloxan with 50 mPa*s | |
| 0.025 g | Inhibitor PTS-I 27 (DVTMDS) from Wacker Chemical | (10.75 mmol Vinyl/g) |
| 19.70 g | Vinylsilicone VS 10,000 from Hanse Chemical, an alpha/omega- | (0.05 mmol Vinyl/g) |
| | Divinylpolydimethylsiloxan with 10, 000 mPa*s | |
| 30.00 g | Silopren Base Mixture P300 from GE Bayer | (0.05 mmol Vinyl/g) |

### Example 3

### “Regular Body & Tensid” (SiH/Vinyl: 1.87)

### Base Paste:

| | | |
|---|---|---|
| 39.00 g | Silopren Base Mixture P1,300 from GE Bayer | (0.06 mmol Vinyl/g) |
| 9.00 g | Silopren Crosslinker 4.3 from GE Bayer | (4.20 rnmol SiH/g) |
| 2.00 g | Silopren Chain Extender TP 3359 from GE Bayer | (1.42 mmol SiH/g) |
| 9.00 g | Vinylsilicone VS 50 from Hanse Chemical, an | (0.63 mmol Vinyl/g) |
| | alpha/omegaDivinylpolydimethylsiloxan with 50 mPa*s | |
| 10.00 g | Vinylsilicone VS 10,000 from Hanse Chemical, an alpha/omega- | (0.05 mmol Vinyl/g) |
| | Divinylpolydimethylsiloxan with 10,000 mPa*s | |
| 30.00 g | Silopren Base Mixture P300 from GE Bayer | (0.05 mmol Vinyl/g) |
| 1.00 g | IGEPAL BC 4 (Rhodia) | |

### Catalyst Paste:

| | | |
|---|---|---|
| 40.00 g | Silopren Base Mixture P1,300 from GE Bayer | (0.06 mmol Vinyl/g) |
| 0.30 g | Catalyst preparation (90 weight % alpha/omega divinylpolydimethylsiloxan with | (0.53 mmol Vinyl/g) |
| | 1,000 mPa*s; 10 weight % 20 catalyst complex “Karstedt”; corr. to 4 weight % pure | |
| | Pt) | |
| 10.00 g | Vinylsilicone VS 50 from Hanse Chemical, an alpha/omega-25 | (0.63 mmol Vinyl/g) |
| | Divinylpolydimethylsiloxan with 50 mPa*s | |
| 0.025 g | Inhibitor PTS-I 27 (DVTMDS) from Wacker Chemical | (10.75 mmol Vinyl/g) |
| 19.70 g | Vinylsilicone VS 10,000 from Hanse Chemical, an alpha/omega- | (0.05 mmol Vinyl/g) |
| | Divinylpolydimethylsiloxan with 10,000 mPa*s | |
| 30.00 g | Silopren Base Mixture P300 from GE Bayer | (0.05 mmol Vinyl/g) |

### 2. Measurement Results

Base paste and catalyst paste of all above-mentioned compositions were homogeneously mixed in equal amounts (50:50), cured and typical features of the resulting masses were determined as follows (if not mentioned otherwise, the methods as outlined herein above were applied for the measurements):

| | | Tear strength | Wetting | Shore | Translucency |
|---|---|---|---|---|---|
| Comp. | consistency | (MPa) | angle | A | (%) |
| Ex. 1 | 43 mm | 2.92 | >90° | 44 | 67.6 |
| | (type 3) | | | | |
| Ex. 2 | 39 mm | 4.76 | >90° | 43 | 67.4 |
| | (type 2) | | | | |
| Ex. 2 | 35 mm | 4.22 | 45° | 43 | 63.3 |
| | (type 2) | | | | |

### Example 4

To show the effect of different fillers regarding translucency the following materials have been prepared and measured:

### REFERENCE (REF): Set 1:1 Mixture of

| Reference Composition (in g) | BASE | CAT |
|---|---|---|
| Silopren Base Mixture P300 | 70.00 | 70.00 |
| SiloprenCrosslinker 4.3 | 9.00 | — |
| Silopren Chain Extender TP 3666 | 2.00 | — |
| Vinylsilicone-VS 50 | 9.00 | 10.00 |
| Vinylsilicone VS 10,000 | 10.00 | 19.62 |
| Inhibitor PTS-I 27 (DVTMDS) | — | 0.03 |
| Catalyst preparation (90 weight % alpha/omega | — | 0.35 |
| divinylpolydimethylsiloxan with 1,000 mPa*s; | | |
| 10 weight % catalyst complex | | |
| “Karstedt”; corr. to 4 weight % pure Pt) | | |
| | 100.00 | 100.00 |

NEGATIVE SAMPLE (NEG): REFERENCE+25% w/w Sikron SF6000 (ground silica, HPF, Frechen, Germany)

### SAMPLE 1 (S1): REFERENCE+25% w/w LiF (99.995%, Sigma-Aldrich, Buchs, Switzerland)

### SAMPLE 2 (S2): REFERENCE+50% w/w LiF (99.995%, Sigma-Aldrich, Buchs, Switzerland)

### SAMPLE 3 (S3): REFERENCE+25% w/w MgF₂ (>99.99%, Sigma-Aldrich, Buchs, Switzerland)

### SAMPLE 4 (S4): REFERENCE+50% w/w MgF₂ (>99.99%, Sigma-Aldrich, Buchs, Switzerland)

| | Add. Filler | RI Filler | Content | Translucency | Hardness |
|---|---|---|---|---|---|
| | Type | n_{D}²³ | in % w/w | in % | in ShoreA |
| REF | none | 1.409* | 0.0 | 57.8 | 50 |
| NEG | Cristobalite | 1.486 | 16.6 | 23.1 | 56 |
| S1 | LiF | 1.40 | 16.6 | 52.2 | 53 |
| S2 | LiF | 1.40 | 33.3 | 46.3 | 57 |
| S3 | MgF2 | 1.39 | 16.6 | 44.7 | 54 |
| S4 | MgF2 | 1.39 | 33.3 | 35.7 | 56 |

*Set silicone material

All compositions according to Examples 1, 2, 3 and 4 proved highly suitable in dental impression taking, as outlined above.

What is claimed is:

1. A polymeric material for taking a dental impression, the polymeric material being curable from an uncured state to a cured state, the polymeric material comprising:
at least one finely dispersed filler;
in the cured state, a translucency in the range of about 30% to about 100%;
in the cured state, a shore A hardness according to DIN 53505:1987 in the range of about 20 to about 70;
in the cured state, a tensile strength according to DIN 53504:1994 in the range of about 1.5 MPa to about 4.5 MPa; and
in the uncured state, a consistency according to DIN ISO 4823:2000 of type 0 to 3.

2. The polymeric material of claim 1, further comprising a silicone-based material.

3. The polymeric material of claim 2, wherein the silicone-based material comprises an addition cross-linked silicone-based material.

4. The polymeric material of claim 2, wherein the silicone-based material comprises a methyl substituted and/or phenyl substituted and/or cyclohexyl-substituted silicone.

5. The polymeric material of claim 1, wherein, at a temperature of 20° C. and at a wavelength of 589 nm, the refractive index of the filler comprises a range of 1.40 to 1.54.

6. The polymeric material of claim 1, wherein the refractive index of the filler at a temperature of 20° C. and at a wavelength of 589 nm differs from the refractive index of the remaining components of the polymeric material at a temperature of 20° C. or at a wavelength of 589 nm by 0.02 or less.

7. The polymeric material of claim 1, wherein the filler comprises a metal fluoride.

8. The polymeric material of claim 1, wherein the metal fluoride is selected from the group consisting of MgF₂, LiF, CaF₂, BaF or any combination thereof.

9. A method of taking a dental impression, the method comprising the step of:
(a) applying a polymeric material at least partially to a region to be reproduced by the dental impression;
(b) identifying an irregularity, the irregularity comprising an air inclusion within the polymeric material or under the translucent polymeric material; and
(c) eliminating the irregularity before hardening of the translucent polymeric material;
wherein the polymeric material is curable from an uncured state to a cured state, the polymeric material comprising
at least one finely dispersed filler;
in the cured state, a translucency in the range of about 30% to about 100%;
in the cured state, a shore A hardness according to DIN 53505:1987 in the range of about 20 to about 70;
in the cured state, a tensile strength according to DIN 53504:1994 in the range of about 1.5 MPa to about 4.5 MPa; and
in the uncured state, a consistency according to DIN ISO 4823:2000 of type 0 to 3.

10. The method of claim 3, wherein, at a temperature of 20° C. and at a wavelength of 589 nm, the refractive index of the filler comprises a range of 1.40 to 1.54.

11. The method of claim 9, wherein the refractive index of the filler at a temperature of 20° C. and at a wavelength of 589 nm differs from the refractive index of the remaining components of the polymeric material at a temperature of 20° C. or at a wavelength of 589 nm by 0.02 or less.

12. The method of claim 9, wherein the filler is a metal fluoride.

13. A kit for taking a dental impression, the kit comprising:
a polymeric material, the polymeric material being curable from an uncured state to a cured state, the polymeric material comprising
at least one finely dispersed filler;
in the cured state, a translucency in the range of about 30% to about 100%;
in the cured state, a shore A hardness according to DIN 53505:1987 in the range of about 20 to about 70;
in the cured state, a tensile strength according to DIN 53504:1994 in the range of about 1.5 MPa to about 4.5 MPa; and
in the uncured state, a consistency according to DIN ISO 4823:2000 of type 0 to 3; and
an additional material which is non-translucent or translucent.

14. The kit of claim 13, wherein, at a temperature of 20° C. and at a wavelength of 589 nm, the refractive index of the filler comprises a range of 1.40 to 1.54.

15. The kit of claim 13, wherein the refractive index of the filler at a temperature of 20° C. and at a wavelength of 589 nm differs from the refractive index of the remaining components of the polymeric material at a temperature of 20° C. or at a wavelength of 589 nm by 0.02 or less.

16. The kit of claim 13, wherein the filler is a metal fluoride.

17. A dental impression comprising:
a cured polymeric material comprising
a translucency in the range of about 30% to about 100%;
a shore A hardness according to DIN 53505:1987 in the range of about 20 to about 70;
a tensile strength according to DIN 53504:1994 in the range of about 1.5 MPa to about 4.5 MPa; and
at least one finely dispersed filler.

18. The dental impression of claim 17, wherein, at a temperature of 20° C. and at a wavelength of 589 nm, the refractive index of the filler comprises a range of 1.40 to 1.54.

19. The dental impression of claim 17, wherein the refractive index of the filler at a temperature of 20° C. and at a wavelength of 589 nm differs from the refractive index of the remaining components of the polymeric material at a temperature of 20° C. or at a wavelength of 589 nm by 0.02 or less.

20. The dental impression of claim 17, wherein the filler is a metal fluoride.
